(19) United States
(12) Patent Application Publication
(10) Pub. No.: US 2006/0263385 A1
(43) Pub. Date: Nov. 23, 2006

(54) Composition to relieve side effects of alcohol intoxication

(76) Inventor: Fran Gare, Aventura, FL (US)

Correspondence Address: ABELMAN, FRAYNE & SCHWAB, 666 THIRD AVENUE, 10TH FLOOR, NEW YORK, NY, 10017, US

(21) Appl. No.: 11/417,994

(22) Filed: May 3, 2006

Related U.S. Application Data
(60) Provisional application No. 60/682,220, filed on May 17, 2005.

(51) Int. Cl. A61K 36/06; A61K 36/28; A61K 31/045; A61K 33/10

(52) U.S. Cl. 424/195.16; 424/687; 424/764; 514/738

(57) ABSTRACT

A composition for the relief of symptoms of acute alcohol intoxication which includes at least one magnesium salt, at least one calcium salt, ascorbic acid, at least one sugar alcohol, at least one liver protecting botanical, at least one probiotic, at least one yeast culture and N-acetyl-L-cysteine.

### FIELD OF THE INVENTION

This invention relates generally to a composition for effecting a reduction in the negative side effects experienced by the human body upon the ingestion of an excess of alcohol. More specifically, the instant invention provides a composition and a method for treating the after-effects of undue alcohol consumption.

### BACKGROUND OF THE INVETION

Since the dawn of time, the negative effects experienced as a result of the excess ingestion of alcohol have been chronicled in fact, fiction and legend. In common parlance, this condition is referred to as a “hangover”. The hallmarks of a hangover are nausea, dizziness, upset stomach, regurgitation, dry mouth, dehydration and, characteristically, a throbbing headache.

Increased liquor consumption, especially binge-type drinking among young adults, college students, and even adolescents, has accentuated the need for a hangover remedy which is safe, cheap, effective, and fast-acting.

Acute alcohol intoxication physiologically results in the body's becoming dehydrated, thus creating an unbalanced state with respect to the minerals and electrolytes found therein, which has proven to be disruptive of the proper biological functioning of the individual. If the level of alcohol ingested is sufficiently high, the liver will not function properly, and the enzymes which function to effect detoxification will, instead, produce toxic metabolites.

The mythic remedies for the treatment of hangover include the administration of aspirin, drinking a Bloody Mary, the drinking of coffee with or without other ingredients, such as orange juice and honey, or simply taking a hot and/or cold shower, as well as countless others. All have proven to be ineffective in alleviating the constellation of debilitating symptoms associated with alcohol-induced hangover.

In addition to these home remedies, there are a number of formulations which require medical prescriptions and often need to be administered by a medical professional. It has been discovered, however, that a number of nutriceuticals, all of which are considered safe for human consumption, possess attributes which address certain of the physiological disruptions to the body and which can be combined without diminishing their collective effectiveness.

Accordingly, it is an object of the present invention to provide a composition which can be safely ingested one or more times after overindulging in alcoholic beverages to either allay and/or to minimize the unpleasant feelings incident to such overindulgence.

It is a further object of the present invention to provide a palliative remedy from alcohol intoxication that would allow an individual to safely and more rapidly return to a state of sobriety without experiencing the morning-after sequalae usually associated with a bout of inebriation.

The foregoing objects of the present invention and other objects will be apparent to those of ordinary skill in the art from the following disclosure.

### SUMMARY OF THE INVENTION

In order to effectuate the foregoing objects, the present invention provides a composition which includes a combination of ingredients which can be dissolved in water and which can be ingested orally to relieve the symptoms of an alcohol-induced hangover and to combat its after-effects by restoring the electrolyte balance, returning the kidney and liver function to normal functioning, neutralizing gastric acidity and relieving headache pain.

### DETAILED DESCRIPTION OF THE INVENTION

The composition, which is preferably in powdered form, includes at least one magnesium salt, at least one calcium salt, ascorbic acid, at least one sugar alcohol, at least one probiotic, at least one yeast source, and N-acetyl-L-cysteine. Optionally, the formulation also includes citric acid and a natural flavoring, such as lemon juice, lime juice, cherry flavoring, etc. Typically, the ingredients which are in powdered form in a sealed packet are added to an 8-ounce glass of water and swallowed.

The composition of the present invention represents a careful balancing of ingredients adapted to the restoration of normal functioning of the liver, kidneys, and stomach, the bodily organs most affected by inebriation, in the shortest time span.

Overindulgence of alcoholic beverages wreaks havoc with blood sugar levels, causing them to drop precipitously, at times so severely so as to produce hypoglycemia and sometimes even a blackout condition. The presence of a magnesium salt, such as magnesium citrate, and a calcium salt, such as calcium carbonate, which are essential mineral and natural antacids, serve to activate certain B vitamins in order to avoid or mitigate the hypoglycemic effect associated with a hangover caused by alcohol, to replace electrolytes and nutrients used up by alcohol in the kidneys and to relieve muscle aches. While from about 300 to about 700 mgs of the magnesium salt and the calcium salt, respectively, can be incorporated advantageously in the present composition, 500 mgs. of each has been found to be preferred from a therapeutic standpoint.

Overconsumption of alcoholic beverages, especially those containing congeners, such as are found in whiskey, can produce toxic levels of acetaldehyde, which vitamin C, also known as ascorbic acid, effectively protects against by stimulating the production of liver detoxifying enzymes. While about 800 to about 1500 mgs of ascorbic acid can be employed to advantage in the present composition, 1250 mgs. has been found to be preferred.

Aggravated stomach upset and intestinal distress are also hallmarks of excess alcohol consumption. This can be remedied by repopulating the intestinal flora with at least one probiotic which contains active bacteria. While bifidus is a preferred species, especially between 300 mgs. and 800 mgs., and, most especially, 500 mgs, L. acidophilus, L. casei, and L. reuteri can all be employed to advantage. In fact, mixtures of the aforementioned probiotics can be employed.

Of the available yeast cultures, Saccharomyces boulardii, is preferred since it replenishes the intestinal flora and improves digestion by causing a rapid multiplication in the number of active bacteria. While 25 mgs. can confer the desired benefit, 50 mgs. or more is preferred for optimal results.

The presence in the composition of at least one botanical, which protects the liver, such as either milk thistle or schisandra, or a mixture of both is also highly desirable since protection is afforded against the toxic effects on liver cells associated with the overconsumption of alcohol and is also supportive of healthy liver functioning. While greater or lesser quantities can be employed to advantage, 50 mgs. has been found to confer the sought-after benefits.

The sugar alcohol, also known as a polyol, present in the formulation, should be at least one selected from the group consisting of xylitol, mannitol, or sorbitol. It sweetens the composition, and provides a pleasant taste and, most importantly, when xylitol is employed, it converts to glutathione in the body which protects the liver and is used by the liver as an essential fatty acid. Xylitol is the preferred sugar alcohol for use in the present invention since it is manufactured by the human body during normal glucose metabolism and is available from other food sources using established energy pathways. While from 750 mgs. to 1,250 mgs. can be employed in the composition of the present invention, 1,000 mgs. is preferred.

N-acetyl-L-cysteine is an amino acid which is present naturally in the human body. It is a precursor in the body to the formation of glutathione, an important antioxidant, which is produced in many cells of the body, particularly liver cells. It is preferred to employ from about 25 mgs. to about 100 mgs. in the composition of the present invention, with 50 mgs. being especially preferred.

Citric acid provides an effervescence to the composition, and natural lemon juice flavor or lime or cherry flavoring provide a pleasant taste which avoids any aftertaste, and their inclusion in the composition is optional. 100 mg. of citric acid is sufficient to produce a satisfactory fizz, while the flavoring agent will provide the desired flavor.

If the after-effects of alcohol overindulgence are not remediated or alleviated completely by the initial dose, a second dose can be taken, also dissolved in about 8 ounces of water, juice, or soda, within about two hours after the initial dose.

The ingredients utilized in the composition of the present invention have been clincially proven safe for ingestion by humans, and their consumption should produce no untoward side effects. Even if the second dose fails to yield the desired results, a third dose may be safely taken, within two hours after the second dose, dissolved in eight to ten ounces of water which should in most instances serve to ameliorate the aforesaid symptoms.

Conveniently, the ingredients in the formulation of the present invention can be provided in a sealed packet or envelope which would comprise a single unit dose. The total of each dose amounts to about 4 to 4.5 grams, depending upon the individual quantities of the ingredients employed.

In another embodiment of the present invention, the formulation or composition can be incorporated in a capsule or tablet and ingested with water or a water-based beverage in which it would readily dissolve.

Although particular embodiments of the invention have been described in detail for the purposes of illustration, various modifications may be made without departing from the spirit and scope of the invention. Accordingly, the invention is not to be limited except as by the appended claims.

What is claimed is:

1. A composition in unit dosage form for oral ingestion by a human in need thereof for relieving the symptoms associated with excessive alcohol consumption, comprising an effective amount of at least one magnesium salt, at least one calcium salt, ascorbic acid, at least one sugar alcohol, at least one botanical, at least one probiotic, at least one yeast culture, and N-acetyl-L-cysteine.

2. The composition of claim 1, wherein the magnesium salt is magnesium citrate.

3. The composition of claim 2, wherein the quantity of magnesium citrate is 500 mgs.

4. The composition of claim 1, wherein the calcium salt is calcium carbonate.

5. The composition of claim 4, wherein the quantity of calcium carbonate is 500 mgs.

6. The composition of claim 1, wherein the quantity of ascorbic acid is 1,250 mg.

7. The composition of claim 1, wherein the sugar alcohol is selected from the gorup consisting of xylitol, mannitol, and sorbitol.

8. The composition of claim 7, wherein the sugar alcohol is xylitol.

9. The composition of claim 8, wherein the quantity of xylitol is 1000 mgs.

10. The composition of claim 1, wherein the botanical is protective of liver function and is selected from the group consisting of milk thistle, schisandra, and mixtures thereof.

11. The composition of claim 10, wherein the quantity of milk thistle is 50 mgs, and the quantity of schisandra is 50 mgs.

12. The composition of claim 1, wherein the probiotic is selected from the group consisting of bifidus, L. acidophilus, L. casei, L. ruteri, and mixtures thereof.

13. The composition of claim 12, wherein the probiotic is bifidus.

14. The composition of claim 13, wherein the quantity of bifidus is 500 mgs.

15. The composition of claim 1, wherein the yeast is saccharomyces boulardii.

16. The composition of claim 15, wherein the quantity of saccharomyces boulardii is 50 mgs.

17. The composition of claim 1, wherein the quantity of N-acetyl-L-cysteine is 50 mgs.

18. A method of relieving the symptoms associated with excessive alcohol consumption by a human in need thereof which comprises ingesting a unit dose of a composition comprising an effective amount of at least one magnesium salt, at least one calcium salt, ascorbic acid, at least one sugar alcohol, at least one botanical, at least one probiotic, at least one yeast culture, and N-acetyl-L-cysteine.

19. The method of claim 18, wherein the magnesium salt is magnesium citrate.

20. The method of claim 19, wherein the quantity of magnesium citrate is 500 mgs.

21. The method of claim 18, wherein the calcium salt is calcium carbonate.

22. The method of claim 21, wherein the quantity of calcium carbonate is 500 mgs.

23. The method of claim 18, wherein the quantity of ascorbic acid is 1,250 mgs.

24. The method of claim 18, wherein the sugar alcohol is selected from the group consisting of xylitol, mannitol, and sorbitol.

25. The method of claim 24, wherein the sugar alcohol is xylitol.

26. The method of claim 25, wherein the quantity of xylitol is 1000 mgs.

27. The method of claim 18, wherein the botanical is protective of liver functioning and is selected from the group consisting of milk thistle, schisandra, and mixtures thereof.

28. The method of claim 27, wherein the quantity of milk thistle is 50 mgs., and the quantity of schisandra is 50 mgs.

29. The method of claim 18, wherein the probiotic is selected from the group consisting of bifidus, L. acidophilus, L. casei, L. ruteri, and mixtures thereof.

30. The method of claim 29, wherein the probiotic is bifidus.

31. The method of claim 30, wherein the quantity of bifidus is 500 mgs.

32. The method of claim 18, wherein the yeast is saccharomyces boulardii.

33. The method of claim 32, wherein the quantity of saccharomyces boulardii is 50 mgs.

34. The method of claim 18, wherein the quantity of N-acetyl-L-cysteine is 50 mgs.
